(19) United States
(12) Patent Application Publication
(10) Pub. No.: US 2004/0156781 A1
(43) Pub. Date: Aug. 12, 2004

(54) Polymeric materials for site specific delivery to the body

(76) Inventors: Christopher H. Porter, Woodenville, WA (US); Robert Ziebol, Blaine, MN (US)

Correspondence Address: Gerald F. Swiss, Foley & Lardner LLP, Three Palo Alto Square, 3000 EI Camino Real, Suite 100, Palo Alto, CA, 94306-2121, US

(21) Appl. No.: 10/686,929

(22) Filed: Oct. 15, 2003

Related U.S. Application Data
(60) Provisional application No. 60/418,251, filed on Oct. 15, 2002.

(51) Int. Cl. A61K 49/04

(52) U.S. Cl. 424/9.4

(57) ABSTRACT

Disclosed are compositions for site specific delivery in the body including diseased vasculature (e.g., aneurysmal sacs, arteriovenous malformations, etc.), body lumens such as the vas deferens and fallopian tubes, cavities created in vivo for the purpose of tissue bulking, and the like. Also disclosed are methods employing such compositions as well as kits comprising such compositions.

### CROSS-REFERENCE TO RELATED APPLICATIONS

This application claims the benefit under 35 U.S.C. § 119(e) of U.S. Provisional Application 60/418,251, filed Oct. 15, 2002, which is hereby incorporated by reference in its entirety.

### BACKGROUND OF THE INVENTION

### FIELD OF THE INVENTION

This invention relates to compositions for site specific delivery in the body including diseased vasculature (e.g., aneurysmal sacs, arteriovenous malformations, etc.), body lumens such as the vas deferens and fallopian tubes, cavities created in vivo for the purpose of tissue bulking and the like. This invention also relates to methods employing such compositions as well as kits comprising such compositions.

The compositions of this invention comprise a non-reactive biocompatible substance and a sufficient amount of a rheological modifier to permit the composition to exhibit thixotropic behavior. This thixotropic behavior permits the compositions to exhibit high viscosities under static conditions while maintaining excellent flow properties under stress.

### REFERENCES

The following publications and patents are cited in this application as superscript numbers:

1. Stoy, Injectable Physiologically Acceptable Polymeric Compositions, International Patent Application Publication No. WO 85/00969, published Mar. 14, 1985

2. Mandai, et al., Direct Thrombosis of Aneurysms with Cellulose Acetate Polymer, J. Neurosurg., 77:497-500 (1992)

3. Whalen II, et al., High Viscosity Embolizing Compositions, U.S. Pat. No. 6,531,111, issued Mar. 11, 2003.

4. Leshchiner, et al., Compositions for Therapeutic Percutaneous Embolization and the Use Thereof, U.S. Pat. No. 5,443,454, issued Jan. 3, 1989

5. Evans, et al., Embolizing Compositions, U.S. Pat. No. 5,695,480, issued Dec. 9, 1997

6. Link, et al., Hydrogel Embolic Agents, Investigative Radiology, 29:746-751 (1994)

7. Young, et al., Vascular Embolotherapy, Vol. 1, Chapter II, Interventional Radiology, pp. 9-32, William & Wilkins, Publishers, (1992)

8. Okada, et al., Intravascular Embolizing Agent Containing Angiogenesis-Inhibiting Substance, U.S. Pat. No. 5,202,352, issued on Apr. 13, 1993.

9. Wallace, et al., Methods for Treating Urinary Incontinence in Mammals, U.S. Pat. No. 6,569,417, issued May 27, 2003.

10. Greff, et al., Methods for Soft Tissue Augmentation in Mammals, U.S. Pat. No. 6,231,613, issued May 15, 2001.

11. Wallace, et al., Methods for Treating Urinary Reflux, U.S. Pat. No. 5,958,444, issued Sep. 28, 1999.

12. Silverman, et al., Method for Treating Gastroesophageal Reflux Disease and Apparatus for Use Therewith, issued May 29, 2001.

13. Bromberg, et al., U.S. Pat. No. 5,939,485, issued Aug. 17, 1999.

14. Cohn, et al., U.S. Pat. No. 6,579,951, issued Jun. 17, 2003.

All of the above publications and patents are herein incorporated by reference in their entirety to the same extent as if each individual publication or patent was specifically and individually indicated to be incorporated by reference in its entirety.

### STATE OF THE ART

Compositions for delivery into the body including body cavities are well known in the art. Such compositions have included non-reactive substances optionally in the presence of a liquid (e.g., solvent) and a contrast agent. Non-reactive substances include biocompatible materials such as biodegradable polymers (e.g., collagen), non-biodegradable polymers (e.g., ethylene-vinyl alcohol copolymers, cellulose acetates, hydrogels, etc.),^{1,2,3} gels⁴ and the like. A summary of such non-reactive substances is provided by Young, et al.⁷

The optional biocompatible solvent can be employed to render the composition more lubricous during delivery and/or to dissolve the non-reactive substance. In the former case, the non-reactive substance is delivered as a solid into, e.g., a body cavity and such solid delivery techniques are disclosed by, for example, Leschiner, et al.⁴ In the latter case, a solution is delivered which solution solidifies in vivo to provide for a solid mass which can act as, e.g., a drug depot, an embolic mass, etc.

One group of such compositions recently receiving extensive evaluations are embolic compositions that, again, are well known in the art. Representative embolic compositions include those found in Mandai, et al.,² Whalen II, et al.,³ Leshchiner, et al.,⁴ Evans, et al.,⁵ and Young et al.⁷ Of these compositions, those showing most promise as embolic agents comprise a non-reactive substance that is insoluble in the body fluid, a solvent which dissolves the substance and which dissipates in the fluids of the body and a contrast agent.³ Such compositions are typically employed for a variety of embolic purposes including the treatment of tumors, the treatment of vascular lesions such as aneurysms, arteriovenous malformations (AVM), arteriovenous fistula (AVF), uncontrolled bleeding and the like.

Embolization of blood vessels is preferably accomplished via catheter techniques that permit the selective placement of the catheter at the vascular site to be embolized. In this regard, recent advancements in catheter technology as well as in angiography now permit neuroendovascular intervention including the treatment of otherwise inoperable lesions. Specifically, development of microcatheters and guide wires capable of providing access to vessels as small as 1 mm in diameter allows for the endovascular treatment of many lesions.

When using embolizing compositions for filling cavities of the body, especially brain aneurysms, it is highly desirable that the filling material, after delivery, not flow out of the cavity. It can be stated that the higher the viscosity of the fluid in the aneurysm, the better or more effective the treatment since complications arising from out flow are mitigated.

The desirability of this high viscosity is offset by the problem of delivering these materials. The materials are necessarily transferred to distant locations through long microcatheters to access the aneurysm. The transport of highly viscous materials through these catheters results in high shear stresses which, in turn, results in very high delivery pressures and requires very robust catheters. In practice, however, robust catheters have thick walls and, accordingly, are not very flexible. The lack of flexibility in the catheter makes the navigation through the vasculature upstream of the aneurysm difficult. Accordingly, the lower the viscosity of the fluid being delivered, the easier and more effective the delivery.

In current treatments, there is a trade-off between the viscosity of the material in the aneurysm and the viscosity of the delivery material. Generally, this tradeoff is resolved by using a material that has some compromise viscosity which provides a composition that is easy to deliver but will effectively cause embolization. Even at this compromise viscosity, the treatment of aneurysms can be difficult.

For example, running or flow of composition from its intended delivery site is of concern as well as the fact that when water insoluble contrast agents are employed, retention of these agents in suspension during delivery from the catheter requires shaking of the composition prior to use coupled with the use of particles of sufficiently small size to mitigate against settling.⁵

Still further, the use of a liquid in the composition poses issues such as compatibility of the delivery devices with the liquid employed, potential side-effects of in vivo use of the liquid as it diffuses into the body, and the like. Ideally, the use of the liquid should be optional and determined by the attending clinician based on the disease to be treated, the condition of the patient and other factors well within the skill of the art.

As to the use of prior art compositions for filling other body cavities, similar problems arise. That is to say that the composition should have a sufficient high viscosity to exhibit site selective placement in the body while at the same time being sufficiently fluid as to permit the clinician to readily deliver the material in vivo. Low viscosity materials can continue to flow when placed in vivo and can result in delivery of the composition to unintended sites. Delivery of solid particles are complicated by their difficulty in passing through the delivery means particularly catheters having very small lumens.

As such, there is an ongoing need to provide a material that has a very high viscosity when it is placed in the body cavity and has a low viscosity while it is being delivered.

### SUMMARY OF THE INVENTION

This invention is directed to novel compositions for site specific delivery into the body such as filling cavities in the body, particularly aneurysms, and methods of treatment related thereto. The compositions of this invention have the particular advantage of exhibiting a high static viscosity such that they exhibit site selective placement in vivo and a low viscosity during delivery to permit injection of these compositions under acceptable delivery pressures.

In one embodiment, this application is directed to a composition comprising a non-reactive biocompatible substance which is insoluble in the blood or other body fluid of a mammal and a sufficient amount of a rheological modifier to permit the composition to exhibit thixotropic behavior.

In a further embodiment, the composition further comprises a contrast agent and/or a biocompatible liquid that is preferably miscible in blood or other body fluid. The biocompatible liquid may act as a solvent and dissolve the non-reactive biocompatible substance and/or the rheological modifier or may act as a lubricous agent. In this latter embodiment, the non-reactive substance is either insoluble or partially soluble in the liquid. In either case, the biocompatible liquid is preferably miscible in the blood or other body fluid such that upon administration in vivo the liquid dissipates leaving a mass of the non-reactive substance in the desired in vivo environment.

The non-reactive biocompatible substance is preferably selected from the group consisting of biocompatible polymers, gels, waxes, beads and lipids.

Examples of biocompatible polymers include biodegradable polymers such as polylactic acid, polyglycolic acid, copolymers of polylactic acid and polyglycolic acid, polyepsilon caprolactone, polyhydroxy butyric acid, polyorthoesters, polyacetals, polydihydropyrans, collagen and mixtures thereof.

Other examples of biocompatible polymers include non-biodegradable polymers such as polyethylene, polypropylene, polybutylene, cellulose acetate, polyethylene terphthalate (PET), polyvinyl chloride, polystyrene, polyamides, nylon, polycarbonates, polysulfides, polysulfones, copolymers including one or more of the foregoing, such as ethylene/vinyl alcohol copolymers.

The rheological modifier that imparts thixotropic behavior to the composition can be selected from the group consisting of non-particulate rheological modifiers, particulate rheological modifiers and mixtures thereof. Examples of particulate rheological modifiers include fumed silica, silicatious earths, bentonite, and mixtures thereof. Examples of non-particulate rheological modifiers include polyacrylates, polyalkenes, polyalkyl oxides, polyamides, polycarbonates, cellulosic polymers and copolymers, polydienes, polyesters, polymethacrylates, polysaccharides, polysiloxanes, polystyrenes, polyurethanes, polyvinyl ethers, polyvinyl esters, and mixtures thereof.

The optional contrast agent employed in the compositions of this invention is either water soluble or insoluble. Examples of water insoluble contrast agents include tantalum, tantalum oxide, tungsten, gold, platinum and barium containing compounds, such as barium sulfate. Examples of water soluble constrast agents include metrizamide, iopamidol, jothalamate sodium, jodomide sodium, and meglumine.

The optional biocompatible liquid is selected relative to its intended purpose. Specifically, if the liquid is employed for the purpose of solublizing the non-reactive substance, the liquid is compatible with and will dissolve the non-reactive substance in the amount employed in the composition. When the non-reactive substance is organic, the liquid is generally an organic solvent such as dimethylsulfoxide, alcohols such as ethanol and aldehydes and ketones, such as acetone.

When the optional biocompatible liquid is employed primarily as a lubricous agent, then solubility of the non-reactive substance in the liquid is not critical and liquids such as water, oils, and the like can be employed.

The compositions of this invention can also comprise other optional components such as plasticizers, surfactants, and the like. Examples of plasticizers include aromatic esters, alkyl esters, phthalate esters, citrate esters, glycerol esters, plant derived oils, animal derived oils, silicone oils, iodinated oils, vitamins A, C, E and acetates and esters thereof, and mixtures thereof.

This invention is also directed to a method for delivering compositions of this invention to mammalian patients. These methods comprise inserting an appropriate delivery device at a targeted site in the patient and then administering via the delivery device a composition of this invention as described above under such conditions that a mass is formed in vivo.

The delivery methods described herein can be employed to embolize blood vessels, to bulk tissue, to provide a depot for drug delivery, and the like.

The compositions described herein can further comprise a radioactive material such that the composition can be used to ablate diseased tissue such as tumors, arteriovenous malformations, and the like. Suitable radioactive materials include, for example, of ⁹⁰yttrium, ¹⁹²iridium, ¹⁹⁸gold, ¹²⁵iodine, ¹³⁷cesium, ⁶⁰cobalt, ⁵⁵cobalt, ⁵⁶cobalt, ⁵⁷cobalt, ⁵⁷magnesium, ⁵⁵iron, ³²phosphorous, ⁹⁰strontium, ⁸¹rubidium, ²⁰⁶bismuth, ⁶⁷gallium, ⁷⁷bromine, 129cesium, ⁷³selenium, ⁷²selenium, 72arsenic, ¹⁰³palladium, ¹²³lead, ¹¹¹Indium, ⁵²iron, ¹⁶⁷thulium, ⁵⁷nickel, ⁶²zinc, ⁶²copper, ²⁰¹thallium and ¹²³iodine.

The compositions can also further comprise a medicament such as an angiogenesis inhibiting compound, a steroidal or non-steroidal anti-inflammatory agent, a thrombotic agent, and the like. The invention also contemplates a method for delivering said composition.

Methods for embolizing a blood vessel are preferably accomplished by delivering via a catheter into a vascular site to be embolized a composition of this invention. Such methods preferably comprise inserting the distal end of the catheter in the selected vascular site, delivering via the catheter a composition comprising a non-reactive biocompatible substance, a sufficient amount of a Theological modifier to permit the composition to exhibit thixotropic behavior, optionally a contrast agent, and/or a biocompatible liquid that is miscible in blood or other body fluid under conditions wherein a mass is formed which embolizes the blood vessel.

Methods for bulking tissue are preferably accomplished by delivering via a delivery device at the tissue site to be bulked a composition of this invention. Such methods preferably comprise inserting the delivery device into the selected tissue, delivering via the device a composition comprising a non-reactive biocompatible substance, a sufficient amount of a rheological modifier to permit the composition to exhibit thixotropic behavior, optionally a contrast agent and/or a biocompatible liquid that is miscible in blood or other body fluid under conditions wherein a mass is formed which bulks the tissue.

Suitable tissue sites for bulking include the suburethral tissue, the periurethreal tissue, soft tissue and sphincters such as the esophageal sphincter.

Suitable delivery devices includes syringes, catheters, and the like.

This invention is also directed to a kit of parts comprising a non-reactive biocompatible substance, a sufficient amount of a rheological modifier to permit the composition to exhibit thixotropic behavior, optionally contrast agent, and optionally a biocompatible solvent that is miscible in blood or other body fluid and a delivery device.

The compositions and methods of this invention provide one or more of the following advantages relative to non-rheologically modified compositions:

i) when a contrast agent is employed, the compositions require little if any shaking prior to use since the rheological modifier acts as a suspending agent;

ii) the high viscosity of the rheologically modified composition under static conditions permits site specific delivery in vivo including improved start-stop characteristics during delivery (the composition will not tend to flow from the delivery device after the pressure has been removed thereby reducing drool) and more uniform and predictable set-up in vivo; and

iii) during shear stress the rheologically modified composition acts as a piston at the interface of this composition and the previously delivered composition, particularly through a catheter or other delivery device, and effectively removes the prior delivered composition from the delivery device with minimal mixing of the two compositions.

Additional advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1 illustrates the Newtonian viscosity characteristics of an embolic composition comprising polyethylene vinyl alcohol copolymer, DMSO and tantalum under different shear stress conditions. FIG. 1 further illustrates the non-Newtonian behavior of this composition when a sufficient amount of fumed silica is added to the composition in order to permit it to exhibit thixotropic behavior.

FIG. 2A, FIG. 2B and FIG. 2C illustrate the delivery of a composition of this invention into an artificial aneurysm and the formation of a solid mass.

### DETAILED DESCRIPTION OF THE INVENTION

As discussed above, this invention is directed to novel compositions for site selective delivery into the body, such as to aneurysms, as well as to methods of treatment related thereto.

Before this invention is described in detail, it is to be understood that unless otherwise indicated this invention is not limited to any particular composition, as such may vary. It is also to be understood that the terminology used herein is for the purpose of describing particular embodiments only and is not intended to limit the scope of the present invention. It must be noted that as used herein and in the claims, the singular forms “a,” “an” and “the” include plural referents unless the context clearly dictates otherwise. In this specification and in the claims which follow, reference will be made to a number of terms which shall be defined to have the following meanings:

The term “biocompatible” means that the material or substance described is non-toxic at the concentrations employed and is substantially non-immunogenic again at the concentrations employed.

The term “non-reactive substance” refers to any biocompatible material which forms a mass in vivo by non-reactive mechanisms. Such non-reactive substances include, by way of example only, biocompatible polymers, biocompatible gels, and biocompatible waxes which are substantially insoluble in blood or other body fluid, i.e., materials that have a solubility in blood or other body fluid of less than 0.01 mg/mL at 37° C. Materials which require reactive mechanisms to effect mass formation in vivo, such as prepolymers, alginates (which cross-link with, e.g. Ca⁺² in vivo to form a mass), 2-component reactive systems and the like, are not included in this definition. Rather, the non-reactive substance forms a mass in vivo by non-reactive mechanisms including, by way of example only, precipitation, phase change, or delivery of the solid mass itself. It will be appreciated that in some cases, the masses of “non-reactive substances” will undergo changes such as hydrolysis, dissolution, and the like over time.

The term “biocompatible contrast agent” or “contrast agent” refers to a biocompatible radiopaque material capable of being monitored during injection into a mammalian subject by, for example, radiography. In the methods of this invention, the contrast agent is preferably water insoluble (i.e., has a water solubility of less than 0.01 mg/ml at 20° C.). Examples of biocompatible water-insoluble contrast agents include tantalum, tantalum oxide, and barium sulfate, each of which is commercially available in the proper form for in vivo use. Other biocompatible water-insoluble contrast agents include gold, tungsten, and platinum. Preferred biocompatible water-insoluble contrast agents are those having an average particle size of about 10 μm or less. Water soluble contrast agents are also suitable for use herein and include, for example, metrizamide, lipidol and the like. Preferably, the biocompatible contrast agent employed does not cause a substantial adverse inflammatory reaction when employed in vivo.

The term “biocompatible polymer” refers to polymers which are substantially insoluble in the body fluid of the mammal. The biocompatible polymer can be either biodegradable or, preferably, non-biodegradable.

Biodegradable polymers are disclosed in the art. Examples of suitable biodegradable polymers include, but are not limited to, linear-chain polymers such as polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, and copolymers, terpolymers, and combinations thereof. Other biodegradable polymers include, for example, gelatin, collagen, etc.

Suitable non-biodegradable biocompatible polymers include, by way of example, cellulose acetates (including cellulose diacetate), ethylene/vinyl alcohol copolymers (EVOH), hydrogels (e.g., acrylics), polyacrylonitrile, polyvinylacetate, cellulose acetate butyrate, nitrocellulose, copolymers of urethane/carbonate, copolymers of styrene/maleic acid, and mixtures thereof.

The particular biocompatible polymer employed is selected relative to the viscosity of the resulting polymer solution, the solubility of the biocompatible polymer in the biocompatible solvent, and the like. For example, in one embodiment the selected biocompatible polymer is soluble in the amounts employed in the selected biocompatible solvent.

Preferred biocompatible polymers are ethylene/vinyl alcohol copolymers. Other preferred polymers include cellulose acetate butyrate, cellulose diacetate, polymethyl methacrylate, polyvinyl acetate, copolymers of urethane and acrylates, and the like.

Ethylene/vinyl alcohol copolymers comprise residues of both ethylene and vinyl alcohol monomers. Small amounts (e.g., less than 5 mole percent) of additional monomers can be included in the polymer structure or grafted thereon provided such additional monomers do not alter the properties of the composition. Such additional monomers include, by way of example only, maleic anhydride, styrene, propylene, acrylic acid, vinyl acetate and the like.

Ethylene/vinyl alcohol copolymers are either commercially available or can be prepared by art-recognized procedures.

As is apparent, the ratio of ethylene to vinyl alcohol in the copolymer affects the overall hydrophobicity/hydrophilicity of the composition which, in turn, affects the relative water solubility/insolubility of the composition as well as the rate of precipitation of the copolymer in an aqueous environment (e.g., blood or tissue). In a particularly preferred embodiment, the copolymers employed herein comprise a mole percent of ethylene of from about 25 to about 60 and a mole percent of vinyl alcohol of from about 40 to about 75. These compositions provide for requisite precipitation rates suitable for use in the methods described therein.

The term “biocompatible gels” refer to materials which are gels under in vivo conditions. The gels may be preformed prior to delivery such as described by Leshchiner, et al.⁴ Alternatively, the gel may be delivered as an aqueous solution which gelatinates in the presence of a physiological or external trigger which induces a phase change from the aqueous phase to the gel phase. Physiological or external triggers include, for example, pH, heat/cold, salt concentrations, and the like. Compositions undergoing transitions from aqueous solutions to gels are well known in the art and are disclosed, for example, by Bromberg, et al.¹³ and Cohn, et al.¹⁴

The term “thixotropic properties” or “thixotropic behavior” refers to the shear thinning capacity of a composition which correlates with a non-Newtonian viscosity relationship such that the composition flows more easily under higher shear rates. Another exemplified behavior would be that of a Bingham plastic. A Bingham plastic is a material that has infinite viscosity when no shear rate is applied but flows once shear rate is applied. Stated another way, the apparent viscosity of the composition decreases with increased shear rate. Compositions under shear or dynamic conditions should exhibit an apparent viscosity of less 10,000 cP at 40° C. and the viscosity under static conditions should be at least 1.5 times over the dynamic viscosity.

The term “biocompatible liquid” refers to a material liquid at least at body temperature of the mammal.

When the biocompatible liquid is employed to dissolve the biocompatible polymer and/or the non-particulate rheological modifier (as defined below), the biocompatible liquid is employed as a solvent and is sometimes described herein as a “biocompatible solvent”. Suitable biocompatible solvents include, by way of example, ethyl lactate, dimethylsulfoxide (DMSO), analogues/homologues of dimethylsulfoxide, ethanol, acetone, and the like. Aqueous mixtures with the biocompatible solvent can also be employed, provided that the amount of water employed is sufficiently small that the dissolved polymer precipitates upon contact with blood or other bodily fluid. Preferably, the biocompatible solvent is dimethylsulfoxide.

When the biocompatible liquid is employed as a lubricous agent, the solubility of the biocompatible polymer and/or rheological modifier is not essential and suitable solvents such as water, oils, emulsions, and the like can be used.

The term “embolizing” refers to a process wherein a material is injected into a blood vessel which, in the case of, for example, aneurysms, fills or plugs the aneurysmal sac and/or encourages clot formation so that blood flow into the aneurysm ceases. In the case of AVMs, a plug or clot is formed to control/reroute blood flow to permit proper tissue perfusion. In the case of a vascular site, the vascular site is filled to prevent blood flow there through. Embolization of the blood vessel is important in preventing and/or controlling bleeding due to lesions (e.g., organ bleeding, gastrointestinal bleeding, vascular bleeding, and bleeding associated with an aneurysm). In addition, embolization can be used to ablate diseased tissue (e.g., tumors, etc.) by cutting off the diseased tissue's blood supply.

The term “encapsulation” as used relative to the contrast agent being encapsulated in the polymer mass, does not infer any physical entrapment of the contrast agent within the mass, much as a capsule encapsulates a medicament. Rather, this term is used to mean that an integral, coherent mass forms which does not separate into individual components.

The term “rheology” refers to the science of flow and deformation of matter, and describes the interrelation between force, deformation, and time.

The term “rheological modifier” as used herein, refers to a component which, when added to a composition, imparts high viscosity to the composition under static conditions, yet permits the composition to flow freely under shear stress. Compositions of this invention may use one or more rheological modifiers, including combinations of rheological modifiers. As used herein, rheological modifiers are generally classified as a non-particulate rheological modifier or a particulate rheological modifier. The preferred rheological modifier is fumed silica.

The term “non-particulate Theological modifier” as used herein, refers to a rheological modifier which can be solubilized or suspended in the biocompatible liquid employed. Non-particulate rheological modifiers include, but are not limited to, polyacrylates, polyalkenes, polyalkyl oxides, polyamides, polycarbonates, cellulosic polymers and copolymers thereof, polydienes, polyesters, polymethacrylates, polysiloxanes, polystyrenes, polyurethanes, polyvinyl ethers, polyvinyl esters, Carbopol, acrylic polymers, cross-linked acrylic polymers, hydroxypropylcellulose, hydroxypropylmethylcellulose, oxidized polyethylene and their copolymers, polyethylene oxide, polyvinylpyrrolidone, associative thickeners, Carrageenan, carboxymethylcellulose, sodium hydroxyethylcellulose, hydroxyethylcellulose, methylcellulose, Guar, Guar derivatives, Locust Bean Gum, Xanthan Gum, and mixtures thereof.

The term “particulate rheological modifier” as used here, refers to a rheological modifier which is mineral-based. Particulate rheological modifiers include, but are not limited to, silacatious earths, bentonite, organoclays, water-swellable clays, such as lapenite, and silicas such as fumed silica and precipitated, calcium carbonate, titanium dioxide, laminate, titanium oxide, zinc oxide, hydroxyappetite, carbon beads, dispersed fiber, magnetic materials and mixtures thereof.

The term “shear stress” refers to the ratio of force to area across, for example, a liquid. The liquid's response to the applied shear stress is to flow. A velocity gradient forms that gives the “shear rate.” The viscosity of the liquid is the ratio of shear stress to shear rate. Newtonian fluids exhibit a linear relationship between shear stress and shear rate, making viscosity independent of the applied shear conditions. Non-Newtonian fluids do not exhibit the linear relationship between shear stress and shear rate. An example would be a Bingham plastic. “Shear-Thinning” or “pseudoplasticity” is a common non-Newtonian flow, where viscosity decreases as shear increases. In a less common non-Newtonian flow, “shear-Thickening” or “dilatancy,” viscosity increases as shear increases. The biocompatible compositions of the instant invention exhibit Pseudoplastic flow.

“Static conditions” as used herein means that the shear rate applied is at most about 1 s⁻¹.

“Surfactants” are those substances which enhance flow and/or aid dispersion by reducing surface tension when dissolved in water or water solutions, or that reduce interfacial tension between two liquids, or between a liquid and a solid. Surfactants also impede the interaction between the rheological modifier and other components of the system. This allows a more fully developed rheological modified system. Surfactants may be anionic, cationic, and nonionic. Surfactants include detergents, wetting agents, and emulsifiers. Suitable cationic surfactants include organic amines and organic ammonium chlorides (e.g., N-tallow trimethylene diamine diolealate and N-alkyl trimethyl ammonium chloride) and the like. Suitable anionic surfactants include, by way of example, sulfosuccinates, carboxylic acids, alkyl sulfonates, octoates, oleates, stearates, and the like. Suitable nonionic surfactants, include by way of example, bridging molecules discussed above, Tritons, Tweens, Spans and the like.

The term “viscosity” refers to a substance's the ratio shearing stress to rate of shear.

Compositions

The biocompatible rheologically-modified compositions described herein are prepared by conventional methods. For illustrative purposes only, compositions comprising a biocompatible polymer (as the non-reactive substance), a Theological modifier, a biocompatible solvent and a contrast agent are described. It is understood that the omission of the contrast agent from the compositions described herein would entail merely eliminating that aspect during preparation. In any event, these compositions are usually prepared by, in a first step, adding sufficient amounts of a biocompatible polymer to the biocompatible liquid. Gentle heating and stirring can be used as necessary to effect dissolution of the non-reactive substance into the solvent and prevent degradation of components. Excessive heating should not be used in order to prevent evaporation of the solvent. When employed, sufficient amounts of contrast agent are then added to the composition at ambient conditions or at moderately elevated temperatures.

After addition of the polymer and contrast agent to the solvent, the rheological modifier is added under ambient conditions, preferably under inert atmosphere, for example, an argon atmosphere. If a particulate rheological modifier is used, the composition is initially stirred at low RPM (less than about 1000 RPM) to wet the surface of the rheological modifier. Once wetted, the stir rate may be increased to a peripheral tip speed of from about 5 m/sec to about 26.5 m/sec. The tip speed should be maintained until no granular material is evidenced in the composition. When non-particulate rheological modifiers are used, the composition need not be stirred at low RPM, as these modifiers and are easily added to the composition.

The initial viscosity of the composition is controlled by the amount of the non-reactive substance employed and/or its molecular weight. For example, high-viscosity compositions which employ low concentrations of polymer can be achieved by the use of very high molecular weight biocompatible polymers (e.g., those with an average molecular weight greater than 250,000). In the alternative, an high-viscosity composition may be achieved with the use a low molecular weight polymer at a high concentration. Such factors are well known in the art and modification of these parameters will be well within the abilities of one of skill in the art.

The viscosity of the composition is then modified by the addition of one or more Theological modifiers or a mixture thereof. The addition of the rheological modifier(s) provides a composition exhibiting a relative decrease in the viscosity under shear stress as compared to its viscosity under static condition.

A particularly preferred rheologically-modified composition comprises a solution of about 3 to about 12 weight percent of biocompatible polymer, about 20 to about 55 weight percent of a contrast agent, preferably 37 to 40 weight percent of contrast agent about 1 to about 12 percent Theological modifier, and the remaining weight percent of the biocompatible solvent. All of the above percentage values are based on the total weight of composition.

Preferably, the compositions are cohesive.

When the non-reactive substance and the Theological modifier are insoluble in the liquid, such compositions can be prepared by admixing the individual components and stirring in the manner described above until a uniform suspension is formed.

When no liquid is employed in the compositions of this invention, the compositions are admixed and stirred under conditions to form a homogeneous mixture.

Other Components

Surfactants can be optionally employed in the biocompatible rheologically-modified composition. When employed, surfactants maintain dispersion of the rheological modifier and the contrast agent in the liquid. Surfactants also impede the interaction between the rheological modifier and other components of the system. This allows for more fully developed rheologically-modified systems.

When surfactants are employed, a preferred biocompatible rheologically-modified composition comprises about 3 to about 12 weight percent of biocompatible polymer, about 20 to about 55 weight percent of a contrast agent, preferably about 37 to about 40 percent of contrast agent, about 1 to about 12 percent rheological modifier, and about 0.1 to about 1.0 weight percent of the Theological modifier is the surfactant, and the remaining weight percent biocompatible solvent. Again, all of the above percentage values are based on the total weight of composition.

Plasticizers may also be included in the composition to allow the composition to be less brittle. Determining the amount of plasticizer is well within the skill of one in the art.

Methods

The compositions described above can then be employed in methods for site specific delivery into the body including filling of body cavities. For example, the compositions described above can then be employed in methods for the catheter assisted intra-vascular embolization of mammalian blood vessels. The methods of this invention are employed at intra-vascular sites wherein preferably blood flow during the embolization process at the vascular site to be treated is attenuated, but not arrested. Attenuation of blood flow arises by placement of the catheter into the vascular site, wherein blood flow therethrough is reduced. For example, a microballoon may be employed to attenuate blood flow. In the methods of this invention, a sufficient amount of the biocompatible rheologically-modified composition is introduced into the vascular site via, for example, a catheter under fluoroscopy so that upon formation of the mass, the vascular site is embolized. The particular amount of composition employed is dictated by the total volume of the vasculature to be embolized, the concentration of polymer in the composition, the rate of mass formation, etc. Such factors are well within the skill of the art.

In the catheter delivery methods described herein, a small diameter medical catheter (i.e., microcatheter) having a diameter typically from about 1 mm to about 3 mm is employed. The particular catheter employed is not critical, provided that catheter components are compatible with the composition (i.e., the catheter components will not readily degrade in the composition). In this regard, it is preferred to use polyethylene in the catheter components because of its inertness in the presence of the composition described herein. Other materials compatible with the compositions can be readily determined by the skilled artisan and include, for example, other polyolefins, fluoropolymers (e.g., polytetrafluoroethylene, perfluoroalkoxy resin, fluorinated ethylene propylene polymers, etc.), silicone, etc. The specific polymer employed is selected relative to stability in the presence of the solvent and preferably has lubricious properties.

Alternatively, the compositions of this invention can be used for tissue bulking or augmentation. For example, injection of the material into the periurethral tissue to form a solid mass can be used to treat incontinence in a manner similar to that described by Wallace, et al.⁹ Further, the compositions of this invention can be used to augment soft tissue in a manner similar to that described by Greff, et al.¹⁰ he compositions of this invention can also be used to augment the suburethral tissue in mammals in order to treat urinary reflux as described by Wallace, et al.¹¹ Augmentation of sphincters can be achieved in a manner similar to that described by Silverman, et al.¹²

Still further, the compositions of this invention can be used for the site specific delivery of a medicament or other material, e.g., a radioactive material, to a selected location in the body. Such medicaments can include anti-angeogenesis materials as described, for example, by Okada, et al.⁸ Other medicaments can include steroidal and non-steroidal anti-inflammatory agents, thrombotic agents and the like. Radioactive materials can be site specific delivered for the ablation of diseased tissue such as tumors, arteriovenous malformations, and the like.

Utility

The compositions and methods described herein are useful for site specific delivery of a composition into a mammalian body. The composition can be used, for instance, in the embolization of mammalian blood vessels which, in turn, can be used to prevent/control bleeding (e.g., organ bleeding, gastrointestinal bleeding, vascular bleeding, bleeding associated with an aneurysm) or to ablate diseased tissue (e.g., tumors, etc.). Accordingly, the invention finds use in human and other mammalian subjects requiring embolization of blood vessels.

The compositions have further utility in bulking soft tissue, sphincters lacking sufficient muscular tone to operate effectively, uretheral and periuretheral tissue and the like.

It is contemplated that the compositions can be employed as a carrier for a compatible, pharmaceutically-active compound wherein this compound is delivered in vivo for subsequent release. Such compounds include, by way of example only, antibiotics, anti-inflammatory agents, chemotherapeutic agents, anti-angiogenic agents, radioactive agents, growth factors and the like.

The following examples are set forth to illustrate the claimed invention and are not to be construed as a limitation thereof.

### EXAMPLES

Unless otherwise stated all temperatures are in degrees Celsius. Also, in these examples and elsewhere, abbreviations have the following meanings:

| | |
|---|---|
| DMSO = | dimethylsulfoxide |
| EH5 = | fumed silica having a surface area of approximately 380 m²/g |
| | (BET) |
| EVOH = | ethylene/vinyl alcohol copolymer |
| g = | gram |
| cP = | centipoise |
| RPM = | revolution per minute |
| mm = | millimeter |
| kg = | kilogram |

Equipment

Unless otherwise indicated, the following equipment was employed in the examples below:

1. Waring Blender (17,900 RPM and 21,300 no-load speed)

2. Viscometer—Brookfield, RVDV II+ (Brookfield Engineering, Middleboro, Mass.)

3. T-bar spindle—Brookfield (Brookfield Engineering, Middleboro, Mass.)

4. Helipath stand—Brookfield (Brookfield Engineering, Middleboro, Mass.)

5. Cowles disperser with a 2 inch blade with variable speed mixer (Morehouse-Cowles, Fullerton, Calif.)

The capillary rheometer used in this invention was constructed in the laboratory; however, a suitable rheometer may be purchased from Qualitest (Ft. Lauderdale, Fla.).

Compositions

The silica used in the examples presented below was obtained from Cabot Corporation. The tantalum is Q2 Grade NRC Capacitor grade tantalum metal powder from HC Starck (Newton, Mass.). The DMSO is USP grade.

### Example 1

The purpose of this example is to demonstrate the preparation of a composition of this invention that is suitable, in one embodiment, for embolizing an aneurysm.

In a beaker, 15 g of EVOH (48 percent ethylene-average molecular weight of approximately 100,000) was added to 150 g of DMSO. The composition was covered and heated to 70° C. for 1.5 hours while stirring at 500 RPM. The heating was continued at the indicated temperature until all of the EVOH was dissolved.

In a blender on low (18,000 RPM), containing the EVOH and DMSO, 88.04 g of tantalum powder was added over a period of one minute. Fumed silica (16.5 g of EH5) was then added into the vortex over approximately 2.5 minutes. After the addition of the last of the silica, the blender was ran for an additional 15 seconds. The blender was then run in the following cycles and the sides were scrapped in between the blending cycles; 1-minute, 1-minute, 1-minute, 2-minutes, 3-minutes, 3-minutes.

The viscosity of the compositions of this invention was tested by pre-warming the viscometer to 37° C. and adding the above composition in the viscometer. In order to allow for equilibrium of the viscometer, the composition sat in the non-running viscometer for 15 minutes. Table I below illustrates the change of viscosity for a sample of a composition of this invention.

TABLE I

| | Shear Rate Applied | Viscosity (cP) |
|---|---|---|
| RPM | (s⁻¹) | at 37° C. |
| 1 | 0.93 | 450 |
| 3 | 2.79 | 367 |
| 10 | 9.3 | 285 |
| 30 | 27.9 | 240 |
| 100 | 93 | 209 |
| 30 | 27.9 | 240 |
| 10 | 9.3 | 285 |
| 3 | 2.79 | 367 |
| 1 | 0.93 | 400 |

The above data demonstrate the shear thinning capacity of the composition under stress. Specifically, at the highest stress (100 RPM) the viscosity of the composition is approximately 40% of that under the lowest stress (1 RPM).

I is noted that even at 1 RPM, the composition is subjected to shear stress. Accordingly, a composition prepared in a manner similar to that described above containing 5.1 percent by weight of the rheological modifier was evaluated under different shear conditions to evaluate its viscosity as compared to a similar composition prepared without the fumed silica rheological modifier.

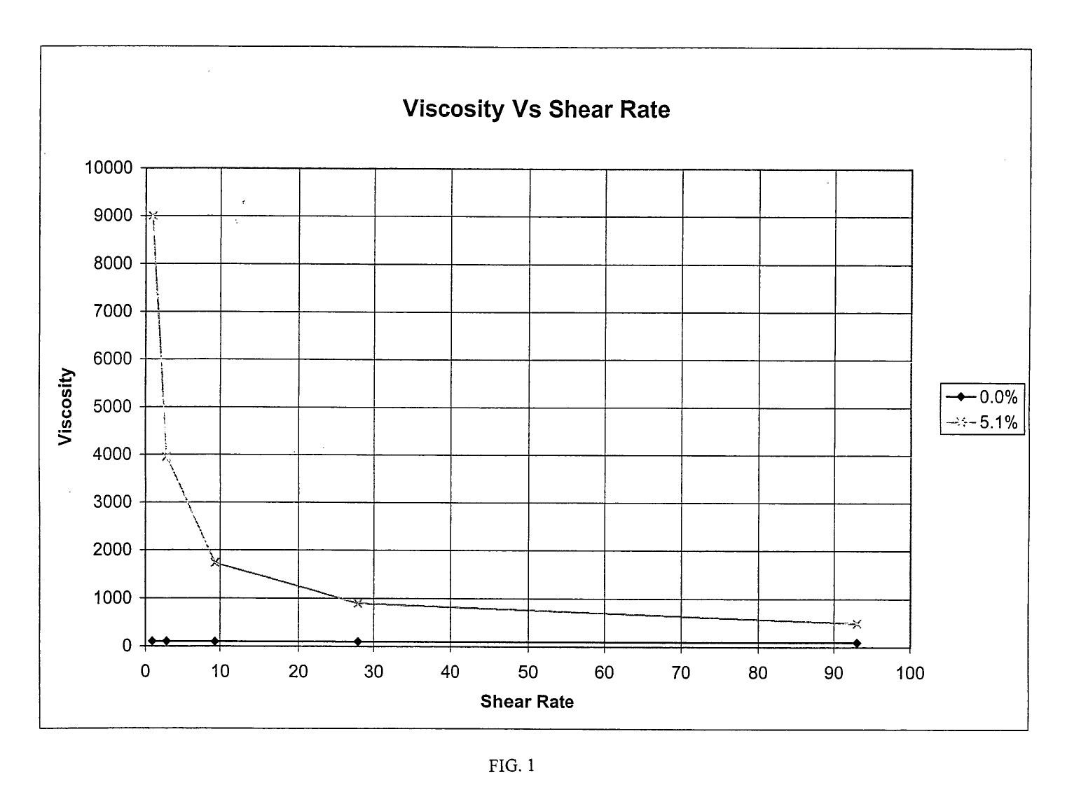

FIG. 1 illustrates that in the absence of the rheological modifier, the composition (depicted by solid diamonds) exhibits Newtonian characteristics. That is to say that the viscosity of the composition does not change with increasing shear rates. Contrarily, FIG. 1 also illustrates that the addition of fumed silica as the rheological modifier provides for a composition exhibiting non-Newtonian characteristics such that the viscosity under high shear rates is significantly less than that under low shear rates. It is this characteristic that provides for facile delivery of the composition while maintaining its property of site specific delivery in vivo.

### Example 2

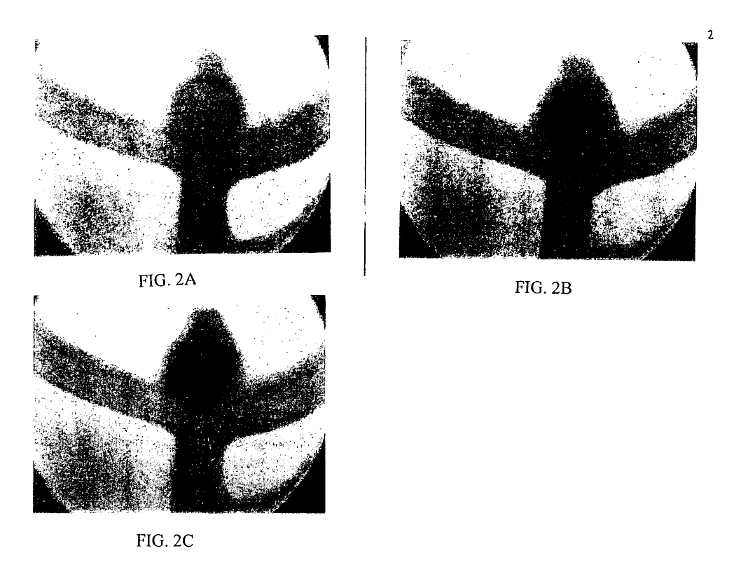

This example illustrates an in vitro application of a Theologically modified embolic composition. This composition was prepared in the manner of Example 1 above and was delivered via a catheter into a Y junction modified to have an artificial aneurysm at the juncture. While a flow of saline was maintained through the Y junction, the distal tip of a catheter was introduced into the artificial aneurysm and the composition was deposited over a time sufficient to fill the aneurysm. As illustrated in FIGS. 2A, 2B and 2C, a solid mass formed in the artificial aneurysm which effectively blocked the aneurysm from the systemic flow.

### Example 3

The purpose of this example is to illustrate how an in vivo application of the composition in the treatment of an aneurysm could be accomplished.

A 10-15 kg mongrel dog is anesthetized. Under sterile conditions and with the aid of an operating microscope, an experimental aneurysm is surgically created in the carotid artery using a jugular vein pouch, employing art recognized protocols. After about one week, the aneurysm is embolized with rheologically-modified composition.

Specifically, the femoral arteries are accessed by cut down and introducers and 7 Fr guiding catheters are placed.

For deposition of the rheologically-modified composition, a microcatheter (e.g., Micro Therapeutics, Inc. Rebar 14, with guide wire) is placed through the guiding catheter and is positioned under fluoroscopic guidance so that the catheter tip is in the aneurysmal sac. A microballoon catheter (4-5 mm balloon) is placed in the carotid artery proximal to the aneurysm. Position is confirmed with injection of a liquid contrast agent. The balloon is inflated to slow or arrest blood flow to prevent displacement of the rheologically-modified composition during injection.

Approximately 0.3 to 0.5 cc of a composition, as described in Example 1, is injected into the aneurysm over 1 to 2 minutes to fill the aneurysm space. Care is given not to overfill the aneurysm and block the parent artery with polymer. Filling is easily visualized with fluoroscopy due to the presence of contrast agent in the polymer composition. After about 5 minutes, the polymer is fully precipitated and the catheters are removed from the artery.

From the foregoing description, various modifications and changes in the composition and method will occur to those skilled in the art. All such modifications coming within the scope of the appended claims are intended to be included therein.

What is claimed is:

1. A composition for placement in a mammalian body comprising:
a) a non-reactive biocompatible substance which is insoluble in blood or other body fluid of a mammal;
b) a sufficient amount of rheological modifier to permit the composition to exhibit thixotropic behavior; and
c) a biocompatible liquid that is miscible in blood or other body fluid.

2. A composition for placement in a mammalian body comprising:
a) a non-reactive biocompatible substance which is insoluble in blood or other body fluid of a mammal;
b) a sufficient amount of rheological modifier to permit the composition to exhibit thixotropic behavior; and
c) a contrast agent.

3. A composition for placement in a mammalian body comprising:
a) a non-reactive biocompatible substance which is insoluble in blood or other body fluid of a mammal;
b) a sufficient amount of Theological modifier to permit the composition to exhibit thixotropic behavior;
c) a biocompatible liquid that is miscible in blood or other body fluid; and
d) a contrast agent.

4. The composition according to claims 1 or 3, wherein the biocompatible liquid is a solvent which dissolves the non-reactive biocompatible substance and/or the rheological polymer.

5. The composition according to claims 1 or 3, wherein the non-reactive substance is either insoluble or partially soluble in the biocompatible liquid.

6. The composition according to claims 1, 2 or 3, wherein the non-reactive substance is selected from the group consisting of biocompatible polymers, gels, waxes, beads and lipids.

7. The composition according to claim 6, wherein the non-reactive substance is a biocompatible polymer.

8. The composition according to claim 7, wherein the biocompatible polymer is a biodegradable polymer.

9. The composition according to claim 8, wherein the biodegradable polymer is selected from the group consisting of polylactic acid, polyglycolic acid, copolymers of polylactic acid and polyglycolic acid, polyepsilon caprolactone, polyhydroxy butyric acid, polyorthoesters, polyacetals, polydihydropyrans, collagen and mixtures thereof.

10. The composition according to claim 7, wherein the biocompatible polymer is a non-biodegradable polymer.

11. The composition according to claim 10, wherein the non-biodegradable polymer is selected from the group consisting of polyethylene, ethylenevinyl alcohol copolymers, cellulose acetate, polypropylene, polybutylene, polyethylene terphthlate, polyvinyl chloride, polystyrene, polyamides, nylon, polycarbonates, polysulfides and polysulfones as well as copolymers, terpolymers of one or more of the foregoing.

12. The composition according to claims 1, 2 or 3, wherein the rheological modifier is selected from the group consisting of non-particulate rheological modifiers, particulate rheological modifiers and mixtures thereof.

13. The composition according to claims 1, 2 or 3, wherein the particulate rheological modifier is selected from the group consisting of silacatious earths, bentonite, organoclays, water-swellable clays, such as lapenite, and silicas such as fumed silica and precipitated, calcium carbonate, titanium dioxide, laminate, titanium oxide, zinc oxide, hydroxyappetite, carbon beads, dispersed fiber, magnetic materials and mixtures thereof.

14. The composition according to claims 1, 2 or 3, wherein the non-particulate rheological modifiers is selected from the group consisting of polyacrylates, polyalkenes, polyalkyl oxides, polyamides, polycarbonates, cellulosic polymers and copolymers thereof, polydienes, polyesters, polymethacrylates, polysiloxanes, polystyrenes, polyurethanes, polyvinyl ethers, polyvinyl esters, Carbopol, acrylic polymers, cross-linked acrylic polymers, hydroxypropylcellulose, hydroxypropylmethylcellulose, oxidized polyethylene and their copolymers, polyethylene oxide, polyvinylpyrrolidone, associative thickeners, Carrageenan, carboxymethylcellulose, sodium hydroxyethylcellulose, hydroxyethylcellulose, methylcellulose, Guar, Guar derivatives, Locust Bean Gum, Xanthan Gum, and mixtures thereof.

15. The composition according to claims 2 or 3, wherein the contrast agent is a water insoluble contrast agent.

16. The composition according to claim 15, wherein the water insoluble contrast agent is selected from the group consisting of tantalum, tantalum oxide, tungsten, gold, platinum and barium sulfate.

17. The composition according to claims 2 or 3, wherein the contrast agent is a water soluble contrast agent.

18. The composition according to claim 17 wherein the water soluble contrast agents is selected from the group consisting of metrizamide, iopamidol, jothalamate socium, jodomide sodium, and meglumine.

19. The composition according to claim 4, wherein the biocompatible liquid is selected from the group consisting of dimethylsulfoxide, ethyl lactate, ethanol and acetone.

20. The composition according to claim 5, wherein the biocompatible liquid is selected from the group consisting of water and oils.

21. The composition according to claims 1, 2 or 3, wherein the composition further comprises one or more agents selected from the group consisting of thickening agents, plasticizers, radioactive agents and surfactants.

22. The composition according to claim 21, wherein the composition comprises further comprises a radioactive agent in a sufficient amount to ablate diseased tissue.

23. The composition according to claim 22, wherein the radioactive material is selected from the group consisting of ⁹⁰yttrium, ¹⁹²iridium, ¹⁹⁸gold, ¹²⁵iodine, ¹³⁷cesium, ⁶⁰cobalt, ⁵⁵cobalt, ⁵⁶cobalt, ⁵⁷cobalt, ⁵⁷magnesium, ⁵⁵iron , ³²phosphorous, ⁹⁰strontium, ⁸¹rubidium, ²⁰⁶bismuth, ⁶⁷gallium, ⁷⁷bromine, ¹²⁹cesium, ⁷³selenium, ⁷²selenium, ⁷²arsenic, ¹⁰³palladium, ²⁰³lead, ¹¹¹indium, ⁵²iron, ¹⁶⁷thulium, ⁵⁷nickel, ⁶²zinc, ⁶²copper, ²⁰¹thallium and ¹²³iodine.

24. The composition according to claim 21, wherein the composition comprises further comprises a medicament.

25. The composition according to claim 24, wherein the medicament is selected from the group consisting of an angiogenesis inhibiting compound, a steroidal or non-steroidal anti-inflammatory agent, and a thrombotic agent.

26. A method for site specific delivery of a composition into a mammalian patient's body which method comprises inserting an appropriate delivery device at a targeted site in the patient and then administering via the delivery device a composition according to any of claims 1-3 under such conditions that a mass is formed in vivo.

27. A method for embolizing a selected vascular site via a catheter having a proximal and distal ends which method comprises inserting the distal end of the catheter in the selected vascular site, delivering via the catheter a composition according to any of claims 1-3 under conditions wherein a solid mass is formed which embolizes the vascular site.

28. A method for bulking tissue via a delivery device having an ejection port which method comprises inserting the ejection port of the delivery device into the tissue to be bulked and delivering via said device a composition according to any of claims 1-3 under conditions wherein a solid mass is formed which bulks the tissue.

29. The method according to claim 28, wherein the tissue targeted for bulking is selected from the group consisting of suburethral tissue, the periurethreal tissue, soft tissue and sphincters such as the esophageal sphincter.

30. A method for delivery of a composition comprising a medicament into a mammalian body which method comprises inserting an appropriate delivery device at a targeted site in the patient and then administering via the delivery device a composition according to claim 24 under such conditions that a mass is formed in vivo.

31. A kit of parts comprising:
a) a composition comprising a non-reactive biocompatible substance, a sufficient amount of a rheological modifier to permit the composition to exhibit thixotropic behavior, optionally contrast agent, and optionally a biocompatible solvent that is miscible in blood or other body fluid; and
b) a delivery device.
